(19) United States
(12) Patent Application Publication
(10) Pub. No.: US 2023/0381427 A1
(43) Pub. Date: Nov. 30, 2023

(54) SYRINGE SEAL

(71) Applicant: Bryna Hansen, Scottsdale, AZ (US)

(72) Inventor: Bryna Hansen, Scottsdale, AZ (US)

(21) Appl. No.: 17/825,853

(22) Filed: May 26, 2022

(51) Int. Cl. A61M 5/50

(52) U.S. Cl. CPC A61M 5/5066; A61M 2005/5073; A61M 2207/10

(57) ABSTRACT

A syringe can comprise a syringe barrel; a syringe plunger; and a syringe seal. The syringe plunger can comprise a plunger body spanning between a plunger proximal end and a plunger distal end, wherein the syringe plunger can be disposed in the syringe barrel such that the plunger proximal end is disposed within the syringe barrel and the plunger distal end is outside the syringe barrel. The syringe seal can comprise and span between a seal first end and a seal second end, wherein the seal first end can be coupled to the syringe barrel and the seal second end can be coupled to the syringe plunger.

### FIELD

This application generally relates to syringes, and more specifically, to syringe seals related to syringe use.

### BACKGROUND

Syringes including needles can be single-use devices, or devices used by a single user. Because hypodermic needles are inserted into a user's body (e.g., through the epidermis), syringe needles are exposed to blood and other tissue of the patient being injected with the substance in the syringe. For safety reasons (e.g., avoiding the spread of disease), hypodermic needs may not be shared between users.

To indicate whether a syringe has been used, there may be a device coupled to the syringe, such as a cap coupled to the syringe plunger portion that is disposed outside of the syringe barrel. Such a cap can be disposed around or over the syringe plunger such that the syringe plunger cannot be moved (rendering the syringe functionally inoperable to collect or dispense a substance for injection). The objective may be to convey to a potential user of the syringe that a syringe having a cap coupled thereto is an unused syringe, and a syringe with the cap decoupled is a used syringe. However, such caps can easily fall off or otherwise become uncoupled from the syringe, thus failing to accurately indicate whether the respective syringe has been previously used. Accordingly, syringes without caps coupled thereto may be determined to be used, and therefore, thrown away, when in fact the syringe was unused.

### SUMMARY OF THE DISCLOSURE

This summary is provided to introduce a selection of concepts in a simplified form. These concepts are described in further detail in the detailed description of example embodiments of the disclosure below. This summary is not intended to necessarily identify key features or essential features of the claimed subject matter, nor is it intended to be used to limit the scope of the claimed subject matter.

The present disclosure relates to syringes and syringes comprising a syringe seal. In various examples, a syringe can comprise a syringe barrel; a syringe plunger; and a syringe seal. The syringe plunger can comprise a plunger body spanning between a plunger proximal end and a plunger distal end, wherein the syringe plunger can be disposed in the syringe barrel such that the plunger proximal end is disposed within the syringe barrel and the plunger distal end is outside the syringe barrel. The syringe seal can comprise and span between a seal first end and a seal second end, wherein the seal first end can be coupled to the syringe barrel and the seal second end can be coupled to the syringe plunger.

In various examples, the seal first end can be melted or welded to the syringe barrel. In various examples, the seal second end can be melted or welded to the syringe plunger. In various examples, the seal first end can comprise a loop configured to be disposed at least partially around the syringe barrel. The loop can comprise a void having a shape that is complementary to a cross-sectional shape of the syringe barrel. In various examples, the seal second end can comprise a clamp, wherein the clamp can be coupled to the syringe plunger. In various examples, the plunger distal end can comprise a plunger flange protruding radially from the plunger body, wherein the clamp can be coupled to the plunger flange. In various examples, the plunger distal end can comprise a plunger flange protruding radially from the plunger body, wherein the seal second end can comprise at least one loop, and wherein the at least one loop can be disposed at least partially around the plunger flange.

In various examples, the syringe seal can be configured such that, in response to the seal first end being coupled to the syringe barrel and the seal second end being coupled to the syringe plunger, the syringe seal may require breaking or decoupling from at least one of the syringe barrel or the syringe plunger in order to translate the syringe plunger within the syringe barrel past a tension point.

In various examples, a method can comprise coupling a seal first end of a syringe seal to a syringe barrel of a syringe, wherein the syringe barrel comprises a barrel body spanning between a barrel needle end and a barrel plunger end; and/or coupling a seal second end of the syringe seal to a syringe plunger of the syringe, wherein the syringe plunger is disposed within the syringe barrel. The syringe seal can be coupled to the syringe barrel and the syringe plunger of the syringe such that the syringe seal may require breaking or decoupling from at least one of the syringe barrel or the syringe plunger in order to translate the syringe plunger within the syringe barrel past a tension point. In various examples, coupling the seal first end to the syringe barrel can comprise coupling the seal first end to the syringe barrel at or proximate the barrel plunger end. In various examples, the syringe barrel at the barrel plunger end can comprise a barrel flange protruding radially from the barrel body. In various examples, coupling the seal first end to the barrel plunger end of the syringe barrel can comprise coupling the seal first end to the barrel flange. In various examples, coupling the seal first end to the syringe barrel can comprise melting or welding, such that seal first end is integral with the syringe barrel. In various examples, coupling the seal second end to the syringe plunger can comprise melting or welding, such that seal second end is integral with the syringe plunger.

In various examples, the seal first end can comprise a loop, wherein coupling the seal first end to the syringe barrel can comprise coupling the loop at least partially around the syringe barrel. In various examples, the syringe barrel at the barrel plunger end can comprise a barrel flange protruding radially from the barrel body. Coupling the seal first end to the syringe barrel can further comprise disposing the loop adjacent to the barrel flange.

In various examples, the syringe plunger can comprise a plunger body spanning between a plunger proximal end configured to be disposed within the syringe barrel and a plunger distal end configured to disposed outside of the syringe barrel. The plunger distal end can comprise a plunger flange protruding radially from the plunger body. Coupling the seal second end to the syringe plunger can comprise coupling the seal second end to the plunger flange. In various examples, the seal second end can comprise a clamp. Coupling the seal second end to the syringe plunger can comprise clamping the seal second end to the plunger flange. In various examples, the method can further comprise breaking the syringe seal between the seal first end and the seal second end to use the syringe.

In various examples, a syringe kit can comprise a syringe barrel; a syringe plunger comprising a plunger body spanning between a plunger proximal end and a plunger distal end; and a syringe seal comprising and spanning between a seal first end and a seal second end. The plunger can be configured to be disposed in the syringe barrel such that the plunger proximal end is disposed within the syringe barrel and the plunger distal end is outside the syringe barrel. The seal first end can be configured to be coupled to the syringe barrel and the seal second end can be configured to be coupled to the syringe plunger.

For the purpose of summarizing the disclosure and the advantages achieved over the prior art, certain objects and advantages of the disclosure have been described herein above. Of course, it is to be understood that not necessarily all such objects or advantages may be achieved in accordance with any particular example of the disclosure. Thus, for example, those skilled in the art will recognize that the examples disclosed herein may be carried out in a manner that achieves or optimizes one advantage or group of advantages as taught or suggested herein without necessarily achieving other objects or advantages as may be taught or suggested herein.

All of these examples are intended to be within the scope of the disclosure. These and other examples will become readily apparent to those skilled in the art from the following detailed description of certain examples having reference to the attached figures, the disclosure not being limited to any particular example(s) discussed.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures. Elements with like element numbering throughout the figures are intended to be the same.

FIG. 1 depicts a syringe system including a syringe seal, in accordance with various examples.

FIG. 2 depicts a perspective view of a syringe system including a syringe seal, in accordance with various examples.

FIGS. 3A and 3B depict perspective views of another syringe system including a syringe seal, in accordance with various examples.

FIGS. 4A and 4B depict perspective views of another syringe system including a syringe seal, in accordance with various examples.

FIG. 4C depicts a side view of the syringe seal included in the syringe system of FIGS. 4A and 4B.

FIG. 5 depicts a perspective view of another syringe system including a syringe seal, in accordance with various examples.

FIGS. 6A and 6B depict perspective views of another syringe system including a syringe seal, in accordance with various examples.

FIG. 7 depicts a method for using a syringe comprising a syringe seal, in accordance with various examples.

### DETAILED DESCRIPTION

All ranges may include the upper and lower values, and all ranges and ratio limits disclosed herein may be combined. It is to be understood that unless specifically stated otherwise, references to “a,” “an,” and/or “the” may include one or more than one, and that reference to an item in the singular may also include the item in the plural, and vice versa.

The detailed description of various examples herein makes reference to the accompanying drawings, which show various examples by way of illustration. While these various examples are described in sufficient detail to enable those skilled in the art to practice the disclosure, it should be understood that other examples may be realized and that logical, chemical, and mechanical changes may be made without departing from the scope of the disclosure. Thus, the detailed description herein is presented for purposes of illustration only and not of limitation. For example, the steps recited in any of the method or process descriptions may be executed in any combination or order and are not necessarily limited to the order or combination presented. Furthermore, any reference to singular includes plural examples, and any reference to more than one component or step may include a singular component or step. Also, any reference to attached, fixed, connected, or the like may include permanent, removable, temporary, partial, full, and/or any other possible attachment option. Additionally, any reference to without contact (or similar phrases) may also include reduced contact or minimal contact.

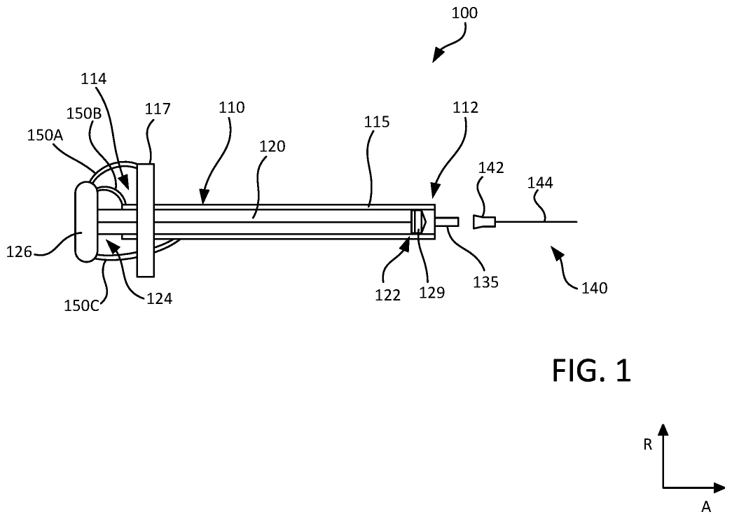

As used herein, the term “aft,” “rear,” or the like refers to the direction associated with the direction a syringe plunger would be translated to collect a substance within the associated syringe barrel. As used herein, the term “forward,” “front,” or the like refers to the direction opposite “aft” or “rear,” and associated with a direction that the substance is dispensed from the needle of a syringe (or the direction in which the syringe needle points). An A-R axis has been included in FIG. 1 to illustrate the axial (A) and radial (R) directions. A circumferential direction may span about an axis (e.g., the axis along which a syringe barrel or plunger spans). For clarity, axial axis A spans parallel to an axis along which at least a portion of a syringe spans. As utilized herein, radially “inward” or “inner” refers to the negative R direction towards the axis along which a syringe spans, and radially “outward” or “outer” refers to the R direction away from such axis.

With reference to FIG. 1, a syringe system 100 (i.e., a syringe 100), in various examples, can comprise a syringe barrel 110, a syringe plunger 120, a syringe tip 135 coupled to syringe barrel 110, a needle 140 coupled to syringe tip 135, and/or a syringe seal 150 coupled to and between syringe barrel 110 and syringe plunger 120. Syringe plunger 120 can be disposed at least partially within syringe barrel 110 and translated axially in syringe barrel 110 to draw a substance through needle 140 and/or syringe tip 135 into syringe barrel 110 and/or to dispense a substance out of syringe barrel 110 through needle 140 and/or syringe tip 135.

In various examples, syringe barrel 110 can comprise a barrel body 115 spanning between a barrel needle end 112 and a barrel plunger end 114. Syringe tip 135 can be coupled to barrel needle end 112. Syringe barrel 110 can span axially (e.g., along the same axis as, or a parallel axis to, the axis along which needle shaft 144 of needle 140 spans). Barrel body 115 can comprise a channel disposed axially therethrough configured to receive syringe plunger 120 within the channel. The channel can be fluidly coupled to a conduit disposed through syringe tip 135. In various examples, syringe barrel 110 can comprise a barrel flange 117 proximate or at barrel plunger end 114. Barrel flange 117 can extend radially from barrel body 115 and be disposed at least partially around an outer surface of barrel body 115. Barrel flange 117 can be configured to provide a user leverage to push syringe plunger 120 in a forward direction (i.e., toward barrel needle end 112) and prevent syringe barrel 110 from sliding in the forward direction in the user's hand. In various examples, a portion of barrel body 115 can be disposed aft of barrel flange 117, or barrel flange 117 can be disposed at the aft-most portion of barrel body 115.

In various examples, needle 140 can comprise needle shaft 144 coupled to and disposed forward of a needle hub 142. Needle hub 142 can couple to syringe tip 135, coupling needle 140 to syringe barrel 110. Needle shaft 144 can be a hypodermic needle with a needle channel disposed therethrough. The needle channel can be in fluid communication with the conduit through syringe tip 135 and the channel disposed through syringe barrel 110.

In various examples, syringe plunger 120 can comprise a plunger body spanning between a plunger proximal end 122 and a plunger distal end 124. At least a portion of syringe plunger 120 can be disposed in syringe barrel 110 (in the channel disposed through barrel body 115). The plunger body can comprise a cross-sectional shape that is complementary to the cross-sectional shape of the channel disposed through barrel body 115. A length of the plunger body can be longer than the associated syringe barrel 110, such that a portion of the plunger body proximate to plunger distal end 124 can be disposed outside of syringe barrel 110 when syringe plunger 120 is fully inserted into syringe barrel 110. Syringe plunger 120 can comprise a piston 129 at or coupled to proximal end 122 configured to seal the portion of the channel within barrel body 115 forward of piston 129 and push any contents therein forward in response to a forward force applied to syringe plunger 120. In various examples, syringe plunger 120 can comprise a plunger flange 126 protruding radially from the plunger body. Plunger flange 126 can be disposed proximate or on plunger distal end 124. Plunger flange 126 can provide a platform upon which a user of syringe 100 can exert force on syringe plunger 120 to translate syringe plunger 120 in the forward direction within syringe barrel 110.

As explained herein, it may be desirable for users to be able to tell whether a syringe has been previously used (to avoid sharing needles, which can cause a risk of disease transmission or other negative consequences to one's health). In various examples, a syringe can comprise a syringe seal, which couples the syringe plunger to the syringe barrel. The syringe seal can be coupled to and between the syringe plunger to the syringe barrel such that the syringe plunger cannot be translated in an aft direction (i.e., further out of the syringe barrel) past a tension point. The tension point can be the axial position of the syringe plunger such that the syringe seal is taut and the syringe plunger can translate aft within the syringe barrel no further without breaking the syringe seal or otherwise decoupling the syringe seal from the syringe barrel and/or the syringe plunger. In various examples, the syringe seal can be configured to prevent the syringe from being usable in any practical way or prevent or decrease the syringe's practicable utility (e.g., preventing the syringe from collecting any measurable amount of a substance to be injected or otherwise dispensed by the syringe). For example, the tension point can be an axial point of the syringe plunger relative to the syringe barrel at which the syringe is unable to collect a measurable amount of a substance (e.g., the amount of substance a syringe is able to collect at the tension point does not reach any measuring device on the syringe barrel, such as graduation marks).

The syringe seal can be coupled to components of a syringe in any suitable manner. For example, with reference to FIG. 1, syringe seal 150A can be coupled to syringe plunger 120 on plunger flange 126 and to syringe barrel 110 on barrel flange 117, spanning therebetween. As another example, syringe seal 150B can be coupled to syringe plunger 120 on plunger flange 126 and syringe barrel 110 on a portion of barrel body 115 aft of barrel flange 117, spanning therebetween. As another example, syringe seal 150C can be coupled to syringe plunger 120 on plunger flange 126 and syringe barrel 110 on a portion of barrel body 115 forward of barrel flange 117, spanning therebetween. In various examples, a syringe seal can be coupled to syringe plunger 120 on the plunger body thereof.

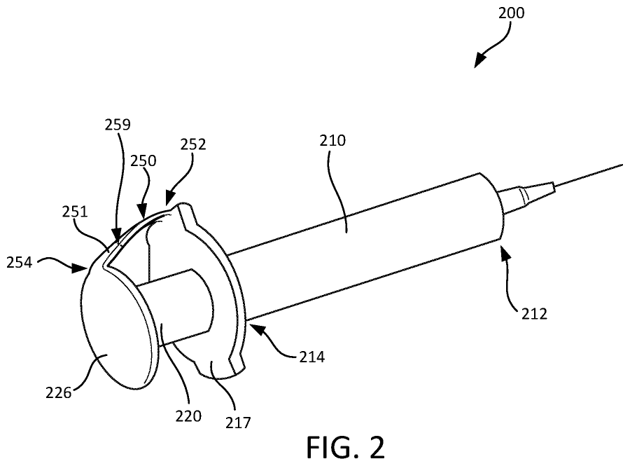

The syringe seal can be coupled to the syringe plunger and syringe barrel of a syringe in any suitable manner. With additional reference to FIG. 2, syringe 200 comprises a syringe barrel 210 with a syringe body spanning between barrel needle end 212 and barrel plunger end 214 (similar to syringe barrel 110 having barrel body 115 spanning between barrel needle end 112 and barrel plunger end 114). Syringe plunger 220 (similar to syringe plunger 120) can be at least partially disposed within syringe barrel 210. In various examples, syringe seal 250 can be coupled to, and/or span between, syringe barrel 210 and syringe plunger 220 via a seal first end 252 of syringe seal 250 being coupled to syringe barrel 210 and a seal second end 254 of syringe seal 250 being coupled to syringe plunger 220. Seal first end 252 can be coupled to barrel flange 217 (similar to barrel flange 117) of syringe barrel 110, or an edge thereof. Seal second end 254 can be coupled to plunger flange 226 of syringe plunger 220, or an edge thereof. Syringe seal 250 can be disposed such that the tension point allows little or no translation of syringe plunger 220 in an aft direction within syringe barrel 210, as discussed herein. Therefore, syringe seal 250 may have to be broken or otherwise decoupled from syringe barrel 210 and/or syringe plunger 220 to practically utilize syringe 200 to collect and dispense a substance.

In various examples, the seal first end can be coupled to the syringe barrel in any suitable manner, and the seal second end can be coupled to the syringe plunger in any suitable manner. In various examples, one or both seal ends can be coupled to the syringe barrel and plunger by an adhesive, clamping, securing (e.g., by a knot, loop, hook, tab, etc.), and/or the like. In various examples, the seal first end and/or seal second end can be fixedly coupled to the syringe barrel or syringe plunger, respectively. As shown in FIG. 2, seal first end 252 can be coupled to syringe barrel 210 (e.g., on barrel flange 217) such that seal first end 252 is integral with syringe barrel 210. In various examples, seal first end 252 can be melted or welded with syringe barrel 210. In various examples, seal first end 252 can be monolithic with syringe barrel 210. In various examples, seal second end 254 can be coupled to syringe plunger 220 (e.g., on plunger flange 226) such that seal second end 254 is integral with syringe plunger 220. In various examples, seal second end 254 can be melted or welded with syringe plunger 220. In various examples, seal second end 254 can be monolithic with syringe plunger 220.

In various examples, a syringe can comprise a seal bridge spanning between and coupled to the seal first and second ends. The seal bridge can be integral and/or monolithic with the seal first and second ends. For example, seal bridge 251 can span between seal first end 252 and seal second end 254 (with regard to syringe seal 250, seal bridge 251 can comprise seal first end 252 and seal second end 254). Seal bridge 251 can be broken, or seal first end 252 and/or seal second end 254 can be decoupled from syringe barrel 210 and/or syringe plunger 220, respectively, to practically utilize syringe 200 to collect or dispense a substance.

In various examples, the seal bridge can have a thickness that partially spans (e.g., circumferentially) around the plunger and/or plunger flange of a syringe. In various examples, the seal bridge can span (or have a thickness of) less than half, less than a third, less than a quarter, less than a tenth, less than a twentieth, less than a fiftieth, or less than a hundredth of the circumferential distance around the plunger and/or plunger flange of a syringe. For example, the thickness of a seal bridge can be less than a centimeter, less than half a centimeter, or less than a quarter centimeter. The thickness of a seal bridge can be any suitable thickness such that the syringe plunger is secured to the syringe barrel and cannot be pulled past a tension point without deliberately breaking the seal bridge or otherwise decoupling at least one of the seal first or second ends from the syringe barrel and/or syringe plunger.

In various examples, a syringe seal can comprise a breakage point. The breakage point can be configured to facilitate the breaking of the syringe seal or seal bridge (or whichever portion of the syringe seal comprises the breakage point) in order to use the syringe. For example, as depicted in FIG. 2, syringe seal 250 can comprise breakage point 259. Breakage point 259 can comprise perforations configured to weaken seal bridge 251 at breakage point 259, such that a user can more easily rip or break seal bridge 251 at breakage point 259. A breakage point can comprise any suitable configuration, such as comprising perforations, or an incision or hole disposed in the syringe seal, a portion of the syringe seal having a smaller thickness or less material than other portions, and/or the like.

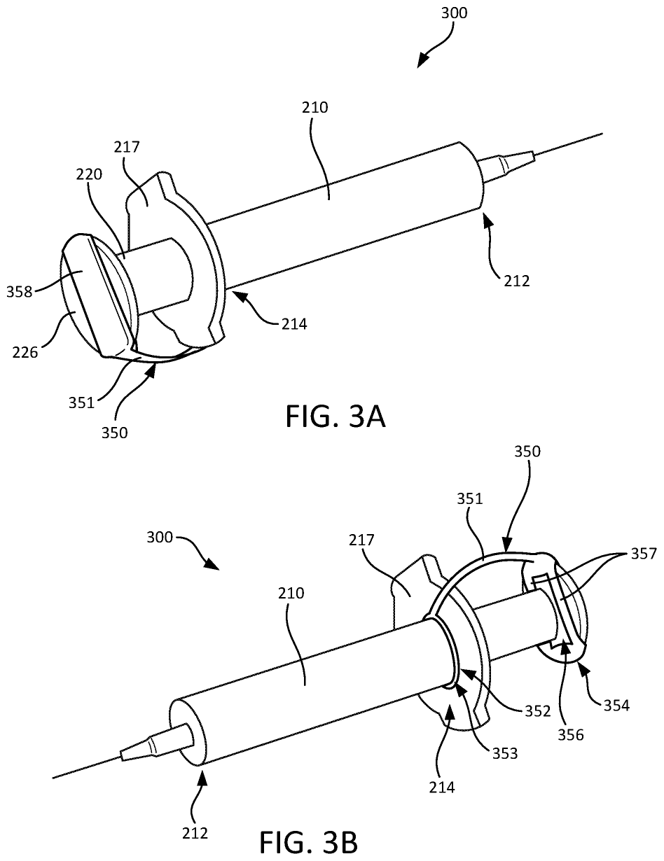

In various examples, one or both of the seal first end or seal second end can be removably coupled to the syringe barrel or syringe plunger, respectively. In various examples, the syringe seal can be a removable component from the rest of the syringe (i.e., non-integral with another component of the syringe). As depicted in FIGS. 3A and 3B, in various examples, a syringe 300 can comprise syringe seal 350 coupled to syringe barrel 210 and syringe plunger 220. Syringe seal 350 can comprise a seal first end 352 comprising a loop 353. Loop 353 can comprise a void having a shape complementary to, and/or configured to receive therein, syringe barrel 210 or a cross-sectional shape thereof. Loop 353 can be disposed at least partially around the barrel body of syringe barrel 210. Syringe seal 350 can comprise seal second end 354. Seal second end 354 can comprise an aft arm 358, which forms at least one loop configured to be disposed at least partially around plunger flange 226 of syringe plunger 220. Aft arm 358 of seal second end 354 can be disposed adjacent to or against an aft surface of plunger flange 226. Aft arm 358 can comprise a length that is complementary or equal to a diameter or length of plunger flange 226. Seal second end 354 can comprise at least one strap, which forms a loop with aft arm 358 configured to be disposed at least partially around plunger flange 226. The strap(s) can be coupled at its ends to aft arm 358. For example, as depicted in FIG. 3B, straps 357 of seal second end 354 can be coupled to aft arm 358 at the ends of each strap 357. Thus, each strap 357 can form a void or loop between the respective strap 357 and aft arm 358. Straps 357 can be disposed around plunger flange 226 on opposing sides of the plunger body of syringe plunger 220. The plunger body can be disposed through void 356 between straps 357. Straps 357 can have a length that is less than a diameter or other dimension of plunger flange 226, which may allow tighter or better securing of syringe seal 350 to plunger flange 226. With straps 357 coupling seal second end 354 to plunger flange 226, seal second end 354 may remain coupled to plunger flange 226 unless deliberately decoupled, thus accurately indicating whether the syringe has been previously used for injection (because syringe seal 350 would be deliberately broken or decoupled from syringe barrel 210 and/or syringe plunger 220 for use).

Syringe seal 350 can comprise seal bridge 351 spanning between and coupled to seal first end 352 and seal second end 354. Seal bridge 351 can be integral and/or monolithic with seal first end 352 and/or seal second end 354. Seal bridge 351 can be broken, or seal first end 352 and/or seal second end 354 can be decoupled from syringe barrel 210 and/or syringe plunger 220, respectively, to practically utilize syringe 300 to collect or dispense a substance.

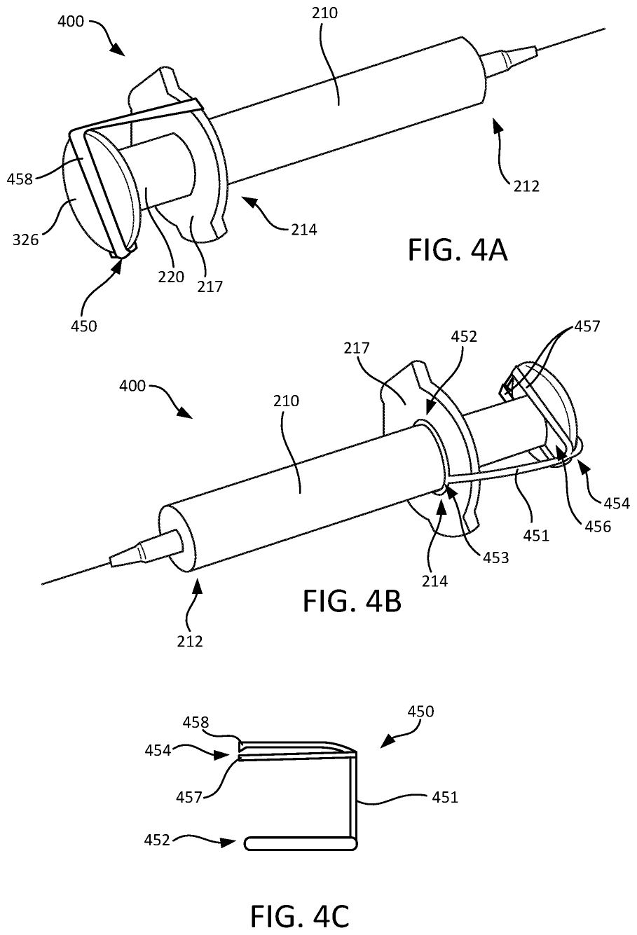

As depicted in FIGS. 4A-4C, in various examples, a syringe 400 can comprise syringe seal 450 coupled to syringe barrel 210 and syringe plunger 220. Syringe seal 450 can comprise a seal first end 452 comprising a loop 453. Loop 453 can comprise a void having a shape complementary to, and/or configured to receive therein, syringe barrel 210 or a cross-sectional shape thereof. Loop 453 can be disposed at least partially around the barrel body of syringe barrel 210. Syringe seal 450 can comprise seal second end 454. Seal second end 454 can comprise an aft arm 458 coupled to seal bridge 451. The end of aft arm 458 opposite of seal bridge 451 can comprise a lip or tab protruding forward. Seal second end 454 can comprise forward arms 457 coupled to seal bridge 451 and disposed forward of aft arm 458. The ends of forward arms 457 opposite of seal bridge 451 can comprise a lip or tab protruding aft from the respective forward arm 457. Seal second end 454 can comprise a clamp comprising aft arm 458 and forward arms 457. The clamp can have a clamping force between aft arm 458 and forward arms 457 caused by aft arm 458 being biased toward forward arms 457, and forward arms 457 being biased toward aft arm 458. Therefore, an object placed with the clamp (i.e., between forward arms 457 and aft arm 458) can be maintained within the clamp by the clamping force of the forward and aft arms.

The clamp can be configured to be coupled, and can be coupled, to the syringe plunger. As shown in FIGS. 4A and 4B, the clamp can be coupled to plunger flange 226. Plunger flange 226 can be disposed within the clamp between aft arm 458 and forward arms 457. Aft arm 458 of seal second end 454 can be disposed adjacent to or against an aft surface of plunger flange 226. Forward arms 457 can be disposed adjacent to or against a forward surface of plunger flange 226. Forward arms 457 can be disposed on opposing sides of the plunger body of syringe plunger 220. The length of the arms of the clamp can be complementary to a dimension of the plunger flange. For example, the clamp, or at least one of the arms thereof, can be complementary to (e.g., equal to or within 10% or 20% of) the diameter of the plunger flange, or the length or width thereof. The lips of the one or more clamp arms, discussed herein, can aid in retaining the plunger flange within the clamp.

Syringe seal 450 can comprise seal bridge 451 spanning between and coupled to seal first end 452 and seal second end 454. Seal bridge 451 can be integral and/or monolithic with seal first end 452 and/or seal second end 454. Seal bridge 451 can be broken, or seal first end 452 and/or seal second end 454 can be decoupled from syringe barrel 210 and/or syringe plunger 220, respectively, to practically utilize syringe 400 to collect or dispense a substance.

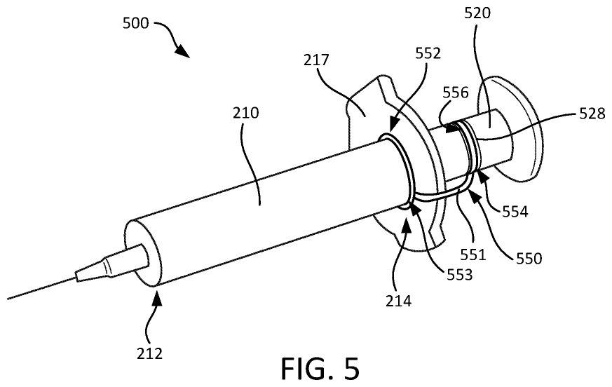

As depicted in FIG. 5, in various examples, a syringe 500 can comprise syringe seal 550 coupled to syringe barrel 210 and syringe plunger 520. Syringe seal 550 can comprise a seal first end 552 comprising a loop 553. Loop 553 can comprise a void having a shape complementary to, and/or configured to receive therein, syringe barrel 210 or a cross-sectional shape thereof. Loop 553 can be disposed at least partially around the barrel body of syringe barrel 210. Syringe seal 550 can comprise seal second end 554. Seal second end 554 can comprise a second loop 556 coupled to seal bridge 551. Second loop 556 can be coupled to syringe plunger 520. In various examples, second loop 556 can be disposed at least partially around syringe plunger 520, e.g., circumferentially around a plunger body of syringe plunger 520. Second loop 556 can comprise a void having a shape complementary to, and/or configured to receive therein, syringe plunger 520 or a cross-sectional shape thereof. In various examples, syringe plunger 520 can comprise a channel 528 recessed into the plunger body at least partially around the plunger body of syringe plunger 520. Channel 528 can be recessed radially inward into the plunger body from an outer edge of the plunger body along at least a portion of a perimeter or circumference of the plunger body. Second loop 556 can comprise a void having a shape complementary to, and/or configured to receive therein, channel 528 or a cross-sectional shape thereof. That is, second loop 556 can be disposed within channel 528. Channel 528 can help to prevent second loop 556 from sliding along the plunger body, which may cause the tension point to change undesirably.

Syringe seal 550 can comprise seal bridge 551 spanning between and coupled to seal first end 552 and seal second end 554. Seal bridge 551 can be integral and/or monolithic with seal first end 552 and/or seal second end 554. Seal bridge 551 can be broken, or seal first end 552 and/or seal second end 554 can be decoupled from syringe barrel 210 and/or syringe plunger 520, respectively, to practically utilize syringe 500 to collect or dispense a substance.

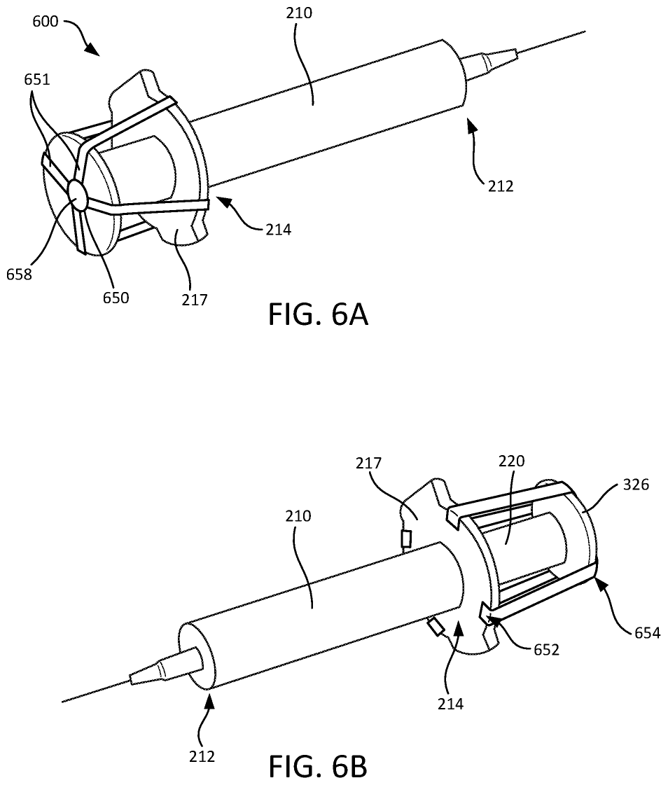

As depicted in FIGS. 6A and 6B, in various examples, a syringe 600 can comprise syringe seal 650 coupled to syringe barrel 210 and syringe plunger 220. Syringe seal 650 can comprise a seal first end 652 and a seal second end 654. Syringe seal 650 can comprise at least one strap 651 coupled to and spanning between syringe barrel 210 and syringe plunger 220. Each strap 651 can comprise part of seal first end 652 and a seal second end 654. In various examples, syringe seal 650 can comprise a seal base 658 coupled to syringe plunger 220 (e.g., on an aft surface of plunger flange 226). At least one strap 651 can be coupled to seal base 658 to couple the strap 651 to syringe plunger 220. An end of the strap(s) 651 opposite of the ends coupled to seal base 658 (i.e., the strap ends of seal first end 652) can be coupled to syringe barrel 210. For example, the strap ends of seal first end 652 can comprise a hook or tab configured to hook or otherwise couple to barrel flange 217. Such tabs can engage a forward edge of barrel flange 217, such that tension of the strap(s) 651 between syringe barrel 210 (and barrel flange 217) and syringe plunger 220 (and plunger flange 226) keeps the strap(s) 651 coupled in place. A syringe seal 650 can comprise any suitable number of straps 652. For example, syringe seal 650 can comprise one strap 652. In other examples, syringe seal 650 can comprise multiple straps 652 that at least partially surround syringe plunger 220 (such as the four straps 651 comprised in syringe seal 650). When there are multiple straps 651, a radial cross-section of the syringe plunger may be imagined as being divided into substantially equal portions equaling the number of straps 652, and each strap 652 can be disposed in a respective portion. For example, as shown in FIG. 6A, there are four straps 652, and each strap 652 can be disposed in its own quarter of the syringe plunger 220 cross-section. That way, tension that facilitates coupling of the syringe seal to the syringe barrel and plunger can be substantially evenly distributed.

In various examples, a syringe seal similar to syringe seal 650 can comprise a rigid material (e.g., a rigid polymeric material, a metal, and/or the like) to maintain coupling of the tabs of straps 652 to barrel flange 217.

In various examples, a syringe seal can be similar to syringe seal 650, but the seal first end 652 can comprise a loop to which the strap(s) 651 couple around the circumference or perimeter of the loop.

In various examples, the components of a syringe (e.g., the syringe barrel, syringe plunger, and syringe seal) can be sold as a kit. In various examples, one or more of the syringe components can be coupled together in the kit.

It should be recognized that any seal first end discussed herein can be paired with any seal second end discussed herein as part of a syringe seal. Additionally, the seal ends of a seal syringe can be coupled in any suitable manner and combination. For example, one seal end can comprise a loop, clamp, hook, tab, and/or the like that couples to the syringe barrel or plunger, and the other seal end can be coupled to the other of the syringe plunger or barrel by adhesive, welding, and/or the like or may be monolithic with the syringe barrel or plunger.

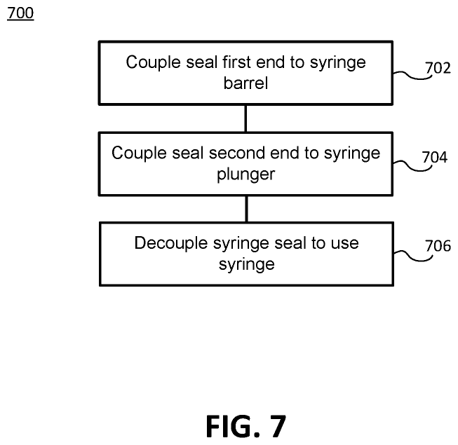

In accordance with various examples, FIG. 7 depicts a method 700 for using a syringe comprising a syringe seal. In various examples, a seal first end can be coupled to a syringe barrel (step 702). As discussed herein, a syringe first end can be coupled to a syringe barrel in any suitable manner. For example, a syringe first end can be welded, melted, adhered, or otherwise coupled to a syringe barrel. As another example, a syringe first end can comprise a structure configured to couple to a syringe barrel such as a loop, clamp, hook, tab, and/or the like.

Continuing with method 700, in various examples, a seal second end can be coupled to a syringe plunger (step 704). As discussed herein, a syringe second end can be coupled to a syringe plunger in any suitable manner. For example, a syringe second end can be welded, melted, adhered, or otherwise coupled to a syringe plunger. As another example, a syringe second end can comprise a structure configured to couple to a syringe plunger such as a loop, clamp, hook, tab, and/or the like.

In response to the seal first end being coupled to the syringe barrel, and the seal second end being coupled to the syringe plunger, the syringe may not be able to be used to collect or dispense a substance because the syringe seal prevents the syringe plunger from translating within the syringe barrel past a tension point. Accordingly, to use the syringe, the syringe seal can be decoupled (step 706). The user can decouple the syringe seal at any suitable location. Such decoupling includes breaking the seal first end, the seal second end, and/or the seal bridge (e.g., at a breakage point). For example, ripping the seal bridge apart severs the coupling between the syringe barrel and the syringe plunger by the syringe seal. As another example, the seal first end can be decoupled from the syringe barrel, and/or the seal second end can be decoupled from the syringe plunger (e.g., by removing the clamp, loop, hook, tab, or other device, or breaking the seal end, from the respective syringe barrel or plunger).

The syringe components can comprise any suitable material, such as a polymeric material, metal material, glass, and/or the like. The syringe seal, specifically, can also comprise any suitable material, such as a polymeric material, metal material, and/or the like. The materials comprised in the syringe seal can be rigid or malleable or flimsy depending on the requirements of the respective configuration and its structure (such as those described herein). Such materials can allow the syringe seal to be effective with minimal material use, inexpensive material and production, and the necessary structural properties.

As discussed herein, a syringe seal allows sealing of a syringe such that the syringe plunger cannot translate past a tension point relative to the syringe barrel. Therefore, the respective syringe cannot be used practically for injection without removal, decoupling, or breaking of the syringe seal from other components of the syringe. Thus, a syringe seal that is intact and coupled to the syringe barrel and syringe plunger can accurately indicate that the syringe is unused (because the removal, breaking, or decoupling of the syringe seal would have to be deliberate, and would not likely just accidentally fall off). Similarly, a syringe seal that is broken and/or decoupled from the syringe barrel and/or syringe plunger can accurately indicate that the syringe is used. Thus, the systems and methods discussed herein can prevent the unknowing sharing of used syringes between patients or users, and also prevent the unknowing waste (e.g., throwing away) of unused syringes caused by users being unsure of whether such syringes were, in fact, previously used.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods, and apparatus are provided herein. In the detailed description herein, references to “one example”, “an example”, “various examples”, etc., indicate that the example described may include a particular feature, structure, or characteristic, but every example may not necessarily include the particular feature, structure, or characteristic. Moreover, such phrases are not necessarily referring to the same example. Further, when a particular feature, structure, or characteristic is described in connection with an example, it is submitted that it is within the knowledge of one skilled in the art to affect such feature, structure, or characteristic in connection with other examples whether or not explicitly described. After reading the description, it will be apparent to one skilled in the relevant art(s) how to implement the disclosure in alternative examples.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method, comprising:
coupling a seal first end of a syringe seal to a syringe barrel of a syringe, wherein the syringe barrel comprises a barrel body spanning between a barrel needle end and a barrel plunger end; and
coupling a seal second end of the syringe seal to a syringe plunger of the syringe, wherein the syringe plunger is disposed within the syringe barrel,
wherein the syringe seal is coupled to the syringe barrel and the syringe plunger of the syringe such that the syringe seal must be at least one of broken or decoupled from at least one of the syringe barrel or the syringe plunger in order to translate the syringe plunger within the syringe barrel past a tension point.

2. The method of claim 1, wherein the coupling the seal first end to the syringe barrel comprises coupling the seal first end to the syringe barrel at or proximate the barrel plunger end.

3. The method of claim 2, wherein the syringe barrel at the barrel plunger end comprises a barrel flange protruding radially from the barrel body.

4. The method of claim 3, wherein the coupling the seal first end to the barrel plunger end of the syringe barrel comprises coupling the seal first end to the barrel flange.

5. The method of claim 1, wherein the coupling the seal first end to the syringe barrel comprises melting or welding, such that seal first end is integral with the syringe barrel.

6. The method of claim 1, wherein the coupling the seal second end to the syringe plunger comprises melting or welding, such that seal second end is integral with the syringe plunger.

7. The method of claim 1, wherein the seal first end comprises a loop, and wherein the coupling the seal first end to the syringe barrel comprises coupling the loop at least partially around the syringe barrel.

8. The method of claim 7, wherein the syringe barrel at the barrel plunger end comprises a barrel flange protruding radially from the barrel body, and wherein the coupling the seal first end to the syringe barrel further comprises disposing the loop adjacent to the barrel flange.

9. The method of claim 1, wherein the syringe plunger comprises a plunger body spanning between a plunger proximal end configured to be disposed within the syringe barrel and a plunger distal end configured to disposed outside of the syringe barrel, wherein the plunger distal end comprises a plunger flange protruding radially from the plunger body, wherein the coupling the seal second end to the syringe plunger comprises coupling the seal second end to the plunger flange.

10. The method of claim 9, wherein the seal second end comprises a clamp, wherein the coupling the seal second end to the syringe plunger comprises clamping the seal second end to the plunger flange.

11. The method of claim 1, further comprising breaking the syringe seal between the seal first end and the seal second end to use the syringe.

12. A syringe, comprising:
a syringe barrel;
a syringe plunger comprising a plunger body spanning between a plunger proximal end and a plunger distal end, wherein the syringe plunger is disposed in the syringe barrel such that the plunger proximal end is disposed within the syringe barrel and the plunger distal end is outside the syringe barrel; and
a syringe seal comprising and spanning between a seal first end and a seal second end, wherein the seal first end is coupled to the syringe barrel and the seal second end is coupled to the syringe plunger.

13. The syringe of claim 12, wherein the seal first end comprises a loop configured to be disposed at least partially around the syringe barrel.

14. The syringe of claim 13, wherein the loop comprises a void having a shape that is complementary to a cross-sectional shape of the syringe barrel.

15. The syringe of claim 12, wherein the seal second end comprises a clamp, wherein the clamp is coupled to the syringe plunger.

16. The syringe of claim 15, wherein the plunger distal end comprises a plunger flange protruding radially from the plunger body, wherein the clamp is coupled to the plunger flange.

17. The syringe of claim 12, wherein the plunger distal end comprises a plunger flange protruding radially from the plunger body, wherein the seal second end comprises at least one loop, and wherein the at least one loop is disposed at least partially around the plunger flange.

18. The syringe of claim 12, wherein the syringe seal is configured such that, in response to the seal first end being coupled to the syringe barrel and the seal second end being coupled to the syringe plunger, the syringe seal must be at least one of broken or decoupled from at least one of the syringe barrel or the syringe plunger in order to translate the syringe plunger within the syringe barrel past a tension point.

19. The syringe of claim 12, wherein at least one of:
the seal first end is at least one of melted or welded to the syringe barrel; or
the seal second end is at least one of melted or welded to the syringe plunger.

20. A syringe kit, comprising:
a syringe barrel;
a syringe plunger comprising a plunger body spanning between a plunger proximal end and a plunger distal end, wherein the syringe plunger is configured to be disposed in the syringe barrel such that the plunger proximal end is disposed within the syringe barrel and the plunger distal end is outside the syringe barrel; and
a syringe seal comprising and spanning between a seal first end and a seal second end, wherein the seal first end is configured to be coupled to the syringe barrel and the seal second end is configured to be coupled to the syringe plunger.
